(19) United States
(12) Patent Application Publication
(10) Pub. No.: US 2020/0113437 A1
(43) Pub. Date: Apr. 16, 2020

(54) SYSTEMS AND METHODS FOR MULTI-MODALITY IMAGING

(71) Applicants: Shenzhen Mindray Bio-Medical Electronics Co., Ltd., Shenzhen (CN); Beijing Shen Mindray Medical Electronics Technology Academy Co., Ltd., Beijing (CN)

(72) Inventors: Donghai Qin, Shenzhen (CN); Fang Yang, Beijing (CN); Lanxi Xiang, Shenzhen (CN); Lei Zhu, Shenzhen (CN)

(21) Appl. No.: 16/392,063

(22) Filed: Apr. 23, 2019

(30) Foreign Application Priority Data
Apr. 23, 2018 (CN) 201810368600.1

(51) Int. Cl. A61B 5/00; A61B 8/14; A61B 8/08; A61B 8/00; A61B 8/06

(52) U.S. Cl. CPC A61B 5/0035; A61B 5/0095; A61B 8/14; A61B 8/488; A61B 8/06; A61B 8/5261; A61B 8/54; A61B 8/085; A61B 8/5246

(57) ABSTRACT

The multi-modality imaging system and method of the present disclosure combine photoacoustic imaging with ultrasound Doppler imaging. When imaging a target tissue, the photoacoustic imaging is used to acquire the functional information of the target tissue and the ultrasound Doppler imaging is used to obtain the magnitude and direction of the flow velocity of the fluid in the target tissue.

### CROSS-REFERENCE TO RELATED APPLICATIONS

This application claims the benefit of priority to Chinese Patent Application No. 201810368600.1, filed on Apr. 23, 2018, which is incorporated herein by reference in its entirety.

### TECHNICAL FIELD

This disclosure relates to ultrasound imaging and in particular to systems and methods for multi-modality imaging.

### BACKGROUND

Ultrasound (US) imaging uses ultrasound beams to scan human tissues and organs and obtains images thereof by receiving and processing reflected signals.

Doppler imaging is a technique that applies the Doppler effect of ultrasound waves reflected or scatted by moving objects. For example, when relative motion exists between the ultrasound source and the reflecting or scatting objects, Doppler shift will occur in received echo signals, and the degree of the Doppler shift will relate to the magnitude and direction of the velocity of the relative motion. Therefore, in medicine, the ultrasonic Doppler imaging technique is used to detect, for example, the magnitude and direction of blood flow velocity in human body.

Although the Doppler imaging technique can also detect the tissue structure or functional information such as vessel depth and inner diameter, it is greatly affected by the angle between blood flow velocity, blood flow direction and the direction of the transmitted ultrasonic wave.

### SUMMARY

The present disclosure provides a multi-modality imaging system and method that addresses the aforementioned problem. In one embodiment, a multi-modality imaging system may include: a laser which emits lasers to a target tissue; a probe which perform transmission and reception of ultrasonic waves to and from the target tissue, wherein the reception of ultrasonic waves comprises receiving photoacoustic signals and receiving ultrasonic echo signals; a controller which controls an emission sequence of the laser and a sequence for transmitting and receiving ultrasonic waves of the probe; and a processor. The processor processes the ultrasonic echo signals to obtain an information for B-mode imaging of the target tissue; analyzes Doppler information of the ultrasonic echo signals to obtain an information for Doppler imaging of the target tissue; processes the photoacoustic signals to obtain an information for photoacoustic imaging of the target tissue; and generates an image of the target tissue according to at least the information for B-mode ultrasonic imaging of the target tissue obtained by the B-mode imaging processing device, the information for Doppler imaging of the target tissue obtained by the Doppler imaging processing device and the information for photoacoustic imaging of the target tissue obtained by the photoacoustic imaging processing device.

In one embodiment, a multi-modality imaging system may include: a laser which emits lasers to a target tissue; a probe which perform transmission and reception of ultrasonic waves to and from the target tissue, wherein the reception of ultrasonic waves comprises receiving photoacoustic signals and receiving ultrasonic echo signals; a controller which controls an emission sequence of the laser and a sequence for transmitting and receiving ultrasonic waves of the probe. The processor analyzes Doppler information of the ultrasonic echo signals to obtain an information for Doppler imaging of the target tissue; processes the photoacoustic signals to obtain an information for photoacoustic imaging of the target tissue; and generates an image of the target tissue according to at least the information for Doppler imaging of the target tissue obtained by the Doppler imaging processing device and the information for photoacoustic imaging of the target tissue obtained by the photoacoustic imaging processing device.

A multi-modality imaging method may include: emitting, by a laser, lasers to a target tissue; receiving, by a probe, photoacoustic signals; transmitting, by the probe, ultrasonic waves to the target tissue to perform a B-mode imaging scanning; receiving, by the probe, ultrasonic echo signals of the B-mode imaging scanning; transmitting, by the probe, ultrasonic waves to the target tissue to perform a Doppler imaging scanning; receiving, by the probe, ultrasonic echo signals of the Doppler imaging scanning; processing, by a processor, the photoacoustic signals to obtain an information for photoacoustic imaging of the target tissue; processing, by the processor, the ultrasonic echo signals of the B-mode imaging scanning to obtain an information for B-mode ultrasonic imaging of the target tissue; analyzing, by the processor, Doppler information of the ultrasonic echo signals of the Doppler imaging scanning to obtain an information for Doppler imaging of the target tissue; and generating, by the processor, an image of the target tissue according to at least the information for photoacoustic imaging of the target tissue, the information for B-mode ultrasound imaging of the target tissue and the information for Doppler imaging of the target tissue.

In one embodiment, a multi-modality imaging method includes: emitting, by a laser, lasers to a target tissue; receiving, by a probe, photoacoustic signals; transmitting, by the probe, ultrasonic waves to the target tissue to perform a Doppler imaging scanning; receiving, by the probe, ultrasonic echo signals of the Doppler imaging scanning; processing, by a processor, the photoacoustic signals to obtain an information for photoacoustic imaging of the target tissue; analyzing, by the processor, Doppler information of the ultrasonic echo signals of the Doppler imaging scanning to obtain an information for Doppler imaging of the target tissue; and generating, by the processor, an image of the target tissue according to at least the information for photoacoustic imaging of the target tissue and the information for Doppler imaging of the target tissue.

### BRIEF DESCRIPTION OF THE DRAWINGS

FIG. 1 is a schematic structural block diagram of a multi-modality imaging system;

FIG. 2 schematically shows the coupling of the laser with the probe through the optical fiber in a multi-modality imaging system;

FIG. 3 schematically shows the photoacoustic scanning and the ultrasonic scanning in a scanning sequence;

FIG. 4A and FIG. 4B schematically show the photoacoustic scanning and the ultrasonic scanning in two scanning sequences;

FIG. 5 is a schematic structural block diagram of a multi-modality imaging system;

FIG. 6 is a schematic structural block diagram of a multi-modality imaging system;

FIG. 7 is a schematic structural block diagram of a multi-modality imaging system including the echo receiving processing device;

FIG. 8 is a flow chart of a multi-modality imaging method;

FIG. 9 is a flow chart for processing the photoacoustic signals in a multi-modality imaging method;

FIG. 10 is a schematic structural block diagram of a multi-modality imaging system;

FIG. 11 schematically shows the photoacoustic scanning and the ultrasonic scanning in a scanning sequence;

FIG. 12 is a schematic structural block diagram of a multi-modality imaging system;

FIG. 13 is a schematic structural block diagram of a multi-modality imaging system; and

FIG. 14 is a flow chart of a multi-modality imaging method.

### DETAILED DESCRIPTION

The present disclosure will further be described in detail by specific embodiments with reference to the drawings below, where similar elements in different embodiments are designated with associated similar reference numbers. In the embodiments below, many details are described in order to make the present disclosure to be better understood. However, a person skilled in the art can easily understand that some of the features may be omitted in some situations or may be replaced by other components, materials or methods. In some cases, relevant operations of the present disclosure are not shown or described in the specification so as to avoid the core of the present disclosure being overwhelmed by too much description. For a person skilled in the art, it is not necessary to describe the relevant operations in detail, because they can fully understand the relevant operations according to the description in the specification and the general technical knowledge in the art.

In addition, the characteristics, operations or features described in the specification may be combined in any suitable ways to form various embodiments. Furthermore, the order of the steps or actions described in the methods may also be changed or adjusted in a manner apparent to those skilled in the art. Therefore, the various orders in the specification and the drawings are only for the purpose of clearly describing a particular embodiment, but not a necessary order, unless it is stated that a certain order must be followed.

References to “first”, “second”, etc., are only used to distinguish the described objects, and do not have any order or technical meaning. As used herein, “connection” or “coupling”, unless otherwise specified, includes both direct and indirect connections (couplings).

Photoacoustic Imaging (PAI) is a new biomedical imaging technology that emerged at the end of the last century. The principle of photoacoustic imaging is based on the photoacoustic effect. When a biological tissue is irradiated by a short pulse (usually on the order of nanoseconds) laser, substances with strong optical absorption properties (such as blood) in the tissue absorb the light energy and cause local heating and thermal expansion, thereby generating ultrasonic signals. Such ultrasonic signals generated by light excitation are generally referred to as a photoacoustic signals. Photoacoustic imaging technology can detect the photoacoustic signals and then reconstruct the position and shape of the absorber in the tissue with high resolution using a corresponding reconstruction algorithm.

What is presented by the photoacoustic imaging is the functional information of an organism. When the photoacoustic imaging is used to detect blood flow, the angle and positional relationship between the probe and the blood vessel need not be considered in the imaging process, but it can only show the position and shape of the blood vessel, while information such as the direction and magnitude (velocity), etc. of the blood flow cannot be presented. Ultrasound Doppler imaging can present information such as the direction and magnitude, etc. of the blood flow, but the tissue structure or function information such as depth of blood vessel and size of inner diameter, etc. presented by the ultrasound Doppler imaging will be greatly affected by the angle between the direction of the blood flow velocity, or the blood flow, and the direction of the ultrasonic wave transmitted by the probe. An ultrasound B image can present the structure and morphology of the tissue surrounding the blood vessel, but the B image itself does not provide information about the blood vessel and the blood flow itself.

Considering these factors, the present disclosure provides a multi-modality imaging system and method that combines photoacoustic imaging with ultrasound Doppler imaging and/or ultrasound B-mode imaging. When imaging a target tissue, the photoacoustic imaging is used to obtain the functional information of the target tissue, while the ultrasound Doppler imaging is used to obtain the magnitude and direction of the velocity of the flow in the target tissue and/or the ultrasound B-mode image is used to obtain the structure and morphology of the tissue surrounding the blood vessel. For example, when imaging a vascular tissue, the photoacoustic imaging is used to obtain the position and shape of the blood vessel, while the ultrasound Doppler imaging is used to obtain the magnitude and direction of the velocity of the blood flow. Thereby, the drawbacks of the photoacoustic imaging and ultrasound Doppler imaging and/or ultrasound B-mode imaging can be overcome, while their advantages can be combined, thereby providing users with more comprehensive and effective information about the target tissue.

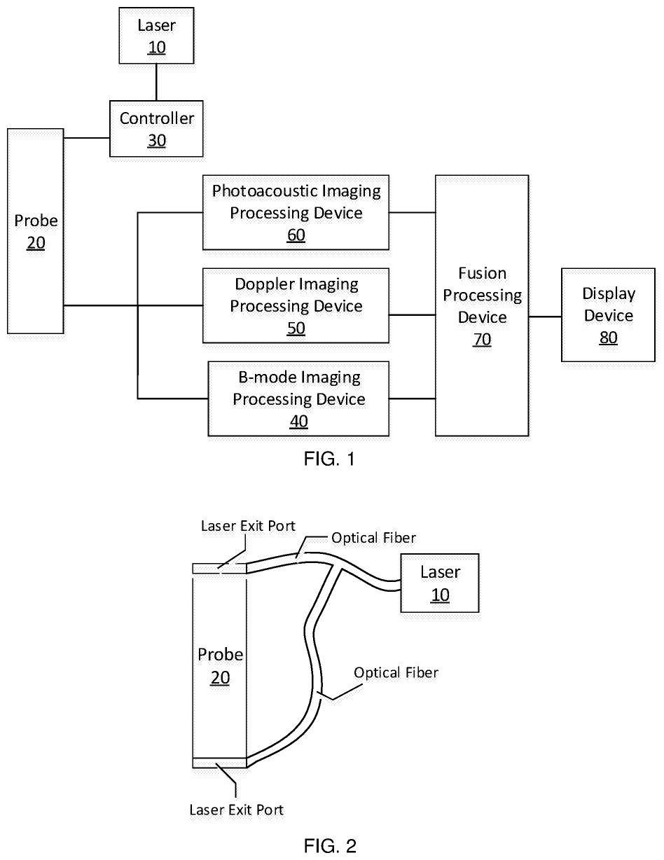

Referring to FIG. 1, in one embodiment, a multi-modality imaging system is provided, which may include a laser 10, a probe 20, a controller 30, a B-mode imaging processing device 40, a Doppler imaging processing device 50, a photoacoustic imaging processing device 60, and a fusion processing device 70. In one embodiment, the multi-modality imaging system may further include a display device 80, as specifically described below.

The laser 10 may generate and transmit lasers, such as lasers with variable wavelength or lasers with fixed wavelength.

The probe 20 may perform transmission and reception of ultrasonic waves to and from a target tissue, where the reception of the ultrasonic waves may include receiving photoacoustic signals and receiving ultrasonic echo signals. In one embodiment, the probe 20 may include an array of elements for converting electrical signals into ultrasonic waves and converting ultrasonic waves into electrical signals, and a laser exit port. The probe 20 may transmit and receive ultrasonic waves by the array of elements described above, where receiving the ultrasonic waves may include receiving photoacoustic signals and receiving ultrasonic echo signals. The laser exit port of the probe 20 may be connected to the laser 10 through an optical fiber so as to transmit the lasers emitted by the laser 10 and transported by the optical fiber. In one embodiment, there may be two laser exit ports which are disposed on both sides of the probe 10, respectively, as shown in FIG. 2.

The controller 30 may control the emission sequence of the laser 10, and the sequence for transmitting and receiving ultrasonic waves of the probe 20. Since the emission frequency of the lasers is low, that is, the time interval between two adjacent laser emissions is relatively long, ultrasonic scans can be inserted therein.

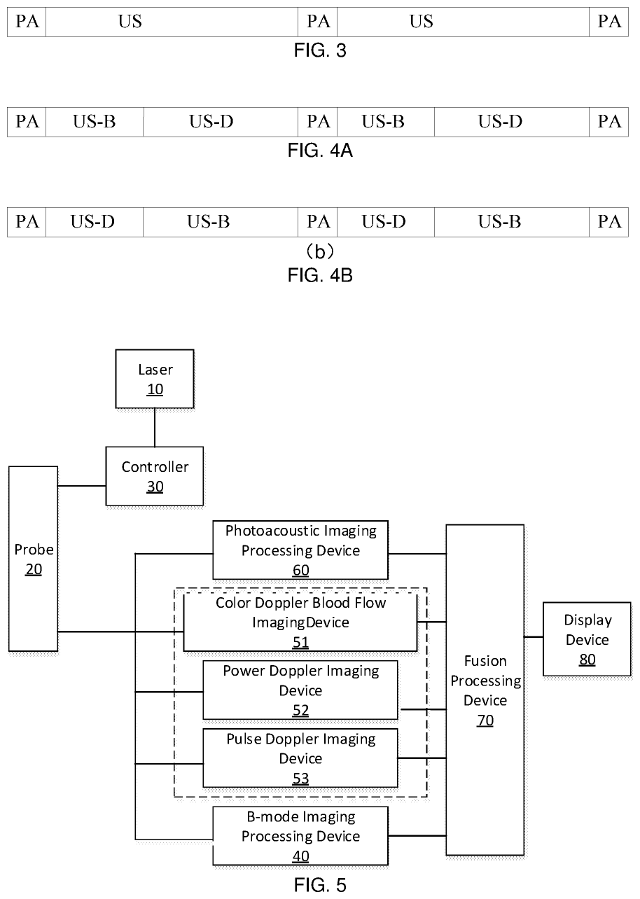

Therefore, as shown in FIG. 3, in one embodiment, the controller 30 may control the laser 10 to emit lasers to a target tissue in a certain sequence, and control the probe 20 to transmit ultrasonic waves to the target tissue during the intermediate period between two adjacent laser emissions. In the figure, PA denotes the emitted laser, and US denotes the ultrasonic scan.

For example, in a scanning control sequence, as shown in FIG. 4A, laser emission, ultrasonic B-mode imaging scanning, and ultrasonic Doppler imaging scanning are performed successively. In a scanning control sequence, as shown in FIG. 4B, laser emission, ultrasonic Doppler imaging scanning and ultrasonic B-mode imaging scanning are performed successively. In FIG. 4A and FIG. 4B, US-B denotes B-mode imaging scanning, and US-D denotes Doppler imaging scanning. Specific description will be presented below.

Taking performing laser emission, ultrasonic B-mode imaging scanning and ultrasonic Doppler imaging scanning successively as an example, in each scanning control sequence, the controller 30 may control the laser 10 to emit laser, and control the probe 20 to receive the photoacoustic signals which will be processed by the photoacoustic imaging processing device 60. The controller 30 may then control the probe 20 to transmit the ultrasonic waves for a B-mode imaging scan, and control the probe 20 to receive the ultrasonic echo signals which will be processed by the B-mode imaging processing device 40. Thereafter, the controller 30 may control the probe 20 to transmit the ultrasonic waves for Doppler imaging scanning, and control the probe 20 to receive the ultrasonic echo signals, which will be processed by the Doppler imaging processing device 50.

Taking the performing of laser emission, ultrasonic Doppler imaging scanning , and ultrasonic B-mode imaging scanning successively as an example, in each scanning control sequence, the controller 30 may control the laser 10 to emit laser, and control the probe 20 to receive the photoacoustic signals which will be processed by the photoacoustic imaging processing device 60. The controller 30 may then control the probe 20 to transmit the ultrasonic waves for Doppler imaging scanning, and control the probe 20 to receive the ultrasonic echo signals which will be processed by the Doppler imaging processing device 50. Thereafter, the controller 30 may control the probe 20 to transmit the ultrasonic waves for B-mode imaging scan, and control the probe 20 to receive the ultrasonic echo signals which will be processed by the B-mode imaging processing device 40.

The B-mode imaging processing device 40 may process the ultrasonic echo signals to obtain information for B-mode imaging of the target tissue.

The Doppler imaging processing device 50 may analyze Doppler information of the ultrasonic echo signals to obtain information for Doppler imaging of the target tissue. In one embodiment, the information for Doppler imaging of the target tissue may include at least motion direction and/or velocity information of the fluid in the target tissue. For example, in the case that the target tissue is vascular tissue, the information for Doppler imaging of the vascular tissue may include at least the direction and magnitude of the velocity of the blood.

Referring to FIG. 5, in one embodiment, the Doppler imaging processing device 50 may include at least one of a color Doppler blood flow imaging device 51, a power Doppler imaging device 52 and a pulse Doppler imaging device 53. It should be noted that in the embodiment shown in the figure all three mentioned above are included. The color Doppler blood flow imaging device 51 is a device which performs ultrasonic imaging using a Color Doppler Flow Image (CDFI) technique, and is used to process ultrasonic echo signals to obtain information for color Doppler flow imaging of the target tissue. The power Doppler imaging device 52 is a device which performs ultrasound imaging using Power Doppler Imaging (PDI) technique, and is used to process ultrasonic echo signals to obtain information for power Doppler imaging of the target tissue. The pulse Doppler imaging device 53 is a device which performs ultrasonic imaging using pulse-wave Doppler Imaging (PWDI) technique, and is used to process ultrasonic echo signals to obtain information for pulse Doppler imaging of the target tissue. Correspondingly, the information for Doppler imaging of the target tissue obtained by the Doppler imaging processing device 50 may include at least one of the information for color Doppler flow imaging of the target tissue, the information for power Doppler imaging of the target tissue and the information for pulse Doppler imaging of the target tissue.

The photoacoustic imaging processing device 60 may process the photoacoustic signals to obtain information for photoacoustic imaging of the target tissue. In one embodiment, the information for photoacoustic imaging of the target tissue may include at least location and/or morphological information of the target tissue. For example, in the case that the target tissue is vascular tissue, the information for photoacoustic imaging of the target tissue may include at least the location and/or morphological information of the blood vessel.

In the photoacoustic imaging processing, the energy value of the emitted laser may need to be known. Since the energy value of each laser emission is different, the laser 10 may be set to record the energy value of the current laser emission after each laser emission is completed. Therefore, the energy value may be read after each time the laser emission is completed. For example, the controller 30 may notify when the energy value may be read.

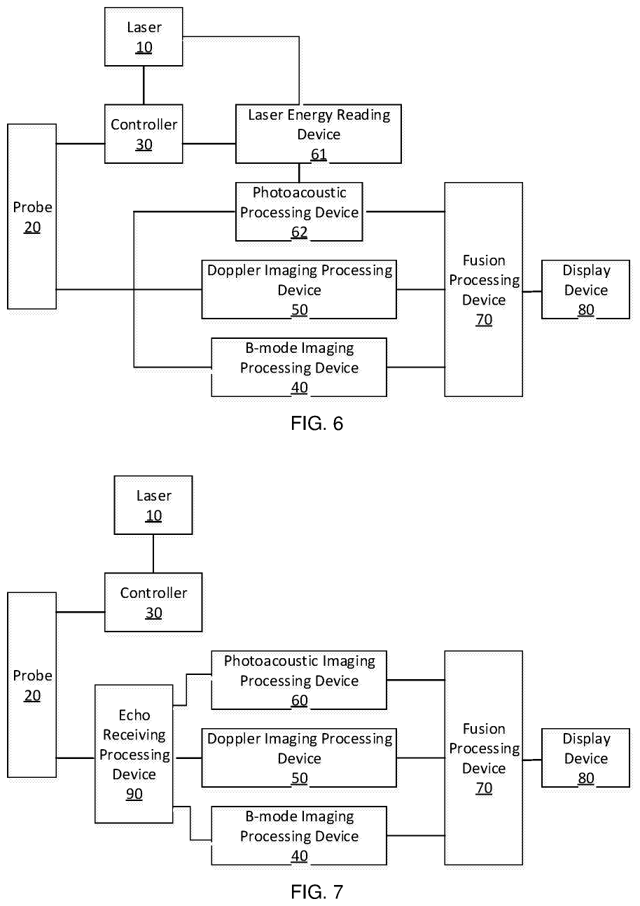

Therefore, in one embodiment, referring to FIG. 6, the photoacoustic imaging processing device 60 may include a laser energy reading device 61 and a photoacoustic processing device 62. The laser 10 may record the energy value of each laser emission, and the laser energy reading device 61 may read the energy value of the each laser emitted by the laser 10. For example, the laser energy reading device 61 may read the energy values recorded by the laser 10 under the control of the controller 30. The photoacoustic processing device 62 may obtain the information for photoacoustic imaging of the target tissue according to the photoacoustic signals and the read energy values of the laser.

The fusion processing device 70 may generate an image of the target tissue according to at least the information for B-mode ultrasonic imaging of the target tissue obtained by the B-mode imaging processing device 40, the information for Doppler imaging of the target tissue obtained by the Doppler imaging processing device 50 and the information for photoacoustic imaging of the target tissue obtained by the photoacoustic imaging processing device 60.

The display device 80 may display the image of the target tissue generated by the fusion processing device 70.

The basic structure of the multi-modality imaging system of the present disclosure is described above, which may also include some other common structural components. For example, referring to FIG. 7, the multi-modality imaging system may further include an echo receiving processing device 90 which processes the signals (such as photoacoustic signals and ultrasonic echo signals) received by the probe 20, such as filtering, amplifying, or beamforming, etc. The echo receiving processing device 90 may send the processed signals to corresponding device. For example, the photoacoustic signal may be processed and sent to the photoacoustic imaging processing device 60, the ultrasonic echo signals obtained by the B-mode imaging scan may be processed and sent to the B-mode imaging processing device 40, and the ultrasonic echo signals obtained by the Doppler imaging scan may be processed and sent to the Doppler imaging processing device 50.

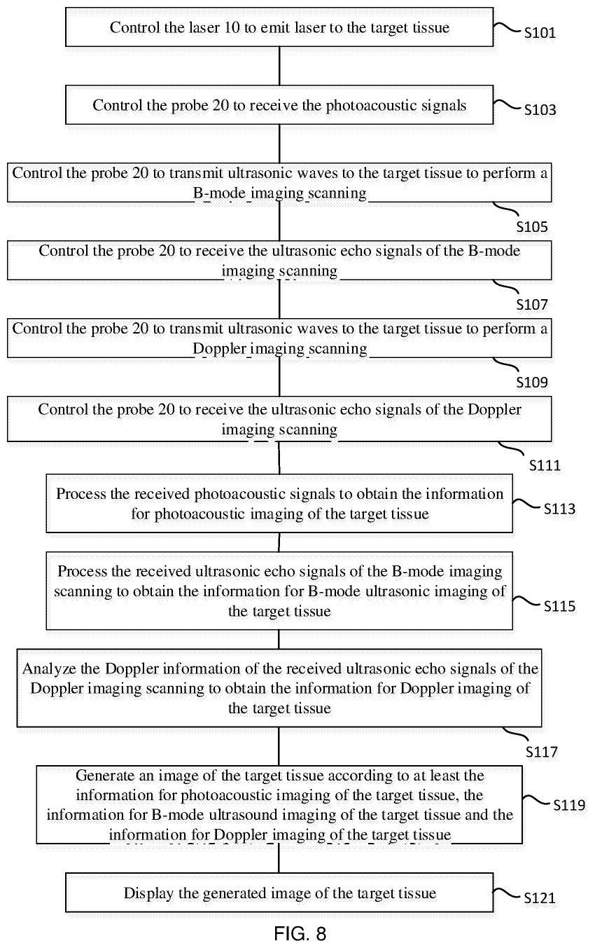

Referring to FIG. 8, in one embodiment, a multi-modality imaging method is provided, which may include steps S101 to S119. In one embodiment, the method may further include step S121. A more specific description will be presented below.

Step S101: controlling the laser 10 to emit laser to the target tissue.

Step S103: controlling the probe 20 to receive the photoacoustic signals.

Step S105: controlling the probe 20 to transmit ultrasonic waves to the target tissue to perform a B-mode imaging scanning.

Step S107: controlling the probe 20 to receive the ultrasonic echo signals of the B-mode imaging scanning.

Step S109: controlling the probe 20 to transmit ultrasonic waves to the target tissue to perform a Doppler imaging scanning.

Step S111: controlling the probe 20 to receive the ultrasonic echo signals of the Doppler imaging scanning.

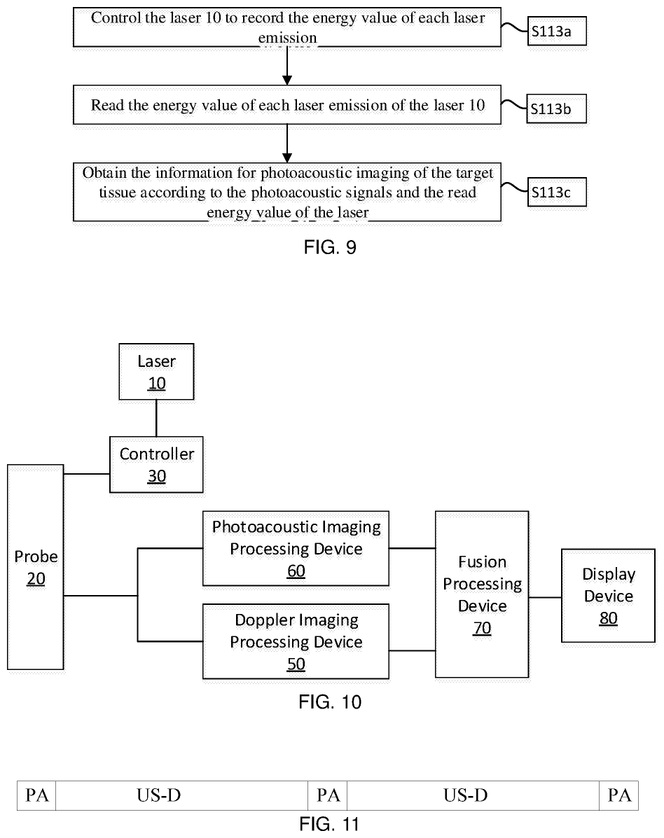

Step S113: processing the photoacoustic signals received in step S103 to obtain the information for photoacoustic imaging of the target tissue. In one embodiment, the information for photoacoustic imaging of the target tissue may include at least location and/or morphological information of the target tissue. Referring to FIG. 9, in one embodiment, step S113 may include steps S113a, S113b, and S113c, which will be specifically described below.

Step S113a: controlling the laser 10 to record the energy value of each laser emission.

Step S113b: reading the energy value of each laser emission of the laser 10. For example, after the laser 10 emits a laser, the energy value of the current laser emission recorded by the laser 10 may be read.

Step S113c: obtaining the information for photoacoustic imaging of the target tissue according to the photoacoustic signals and the read energy value of the laser.

Step S115: processing the ultrasonic echo signals of the B-mode imaging scanning received in step 107 to obtain the information for B-mode ultrasonic imaging of the target tissue.

Step S117: analyzing the Doppler information of the ultrasonic echo signals of the Doppler imaging scanning received in step S111 to obtain the information for Doppler imaging of the target tissue. In one embodiment, the information for Doppler imaging of the target tissue may include at least motion direction and/or velocity information of the fluid in the target tissue. In one embodiment, the information for Doppler imaging of the target tissue may include at least one of: the information for color Doppler flow imaging of the target tissue, the information for power Doppler imaging of the target tissue and the information for pulse Doppler imaging of the target tissue.

Step S119: generating an image of the target tissue according to at least the information for photoacoustic imaging of the target tissue, the information for B-mode ultrasound imaging of the target tissue and the information for Doppler imaging of the target tissue.

Step S121: displaying the image of the target tissue generated in step S119.

In the above steps, steps S101, S105 and S109 involve laser or ultrasonic emission, and steps S103, S107 and S111 involve reception of photoacoustic signals or ultrasonic echo signals. In one embodiment, the sequence of these steps may be controlled. For example, the laser 10 may be controlled to emit lasers to the target tissue in a certain sequence, and the probe 20 may be controlled to transmit ultrasonic waves to the target tissue during an intermediate period between two adjacent laser emissions. The laser 10 may be controlled to emit lasers to the target tissue, and the probe 20 may be controlled to receive the photoacoustic signals; and then, the probe 20 may be controlled to transmit ultrasonic waves to the target tissue to perform B-mode imaging scanning, and receive the ultrasonic echo signals; and thereafter, the probe 20 may be controlled to transmit ultrasonic waves to the target tissue to perform Doppler imaging scanning, and receive the ultrasonic echo signals. Alternatively, the laser 10 may be controlled to emit lasers to the target tissue, and the probe 20 may be controlled to receive the photoacoustic signals; and then, the probe 20 may be controlled to transmit ultrasonic waves to the target tissue to perform Doppler imaging scanning, and receive the ultrasonic echo signals; and thereafter, the probe 20 may be controlled to transmit ultrasonic waves to the target tissue to perform B-mode imaging scanning, and receive the ultrasonic echo signals.

Referring to FIG. 10, in one embodiment, a multi-modality imaging system is provided, which may include a laser 10, a probe 20, a controller 30, a Doppler imaging processing device 50, a photoacoustic imaging processing device 60, and a fusion processing device 70. In one embodiment, the multimodal imaging system may also include a display device 80, as specifically described below.

The laser 10 may generate and transmit lasers, such as lasers with variable wavelength or lasers with fixed wavelength.

The probe 20 may perform transmission and reception of ultrasonic waves to and from a target tissue, where the reception of the ultrasonic waves may include receiving photoacoustic signals and receiving ultrasonic echo signals. In one embodiment, the probe 20 may include an array of elements for converting electrical signals into ultrasonic waves and converting ultrasonic waves into electrical signals, and a laser exit port. The probe 20 may transmit and receive ultrasonic waves by the array of elements described above, where receiving the ultrasonic waves may include receiving photoacoustic signals and receiving ultrasonic echo signals. The laser exit port of the probe 20 may be connected to the laser 10 through an optical fiber so as to transmit the lasers emitted by the laser 10 and transported by the optical fiber. In one embodiment, there may be two laser exit ports respectively disposed on both sides of the probe 10, as shown in FIG. 2 above.

The controller 30 may control the emission sequence of the laser 10, and the sequence for transmitting and receiving ultrasonic waves of the probe 20. Since the emission frequency of the lasers is low, that is, the time interval between two adjacent laser emissions is relatively long, ultrasonic scans can be inserted therein. Therefore, as shown in FIG. 3, in one embodiment, the controller 30 may control the laser 10 to emit lasers to a target tissue in a certain sequence, and control the probe 20 to transmit ultrasonic waves to the target tissue during the intermediate period of two adjacent laser emissions. In the figure, PA denotes the emitted laser, and US denotes the ultrasonic scan.

For example, in a scanning control sequence, as shown in FIG. 11, laser emission and ultrasonic Doppler imaging scanning are performed successively, where US-D denotes the Doppler imaging scanning. Specifically, in a scanning control sequence, the controller 30 may control the laser 10 to emit lasers and control the probe 20 to receive the photoacoustic signals which will be processed by the photoacoustic imaging processing device 60, and thereafter, the controller 30 may control the probe 20 to transmit ultrasonic waves to perform Doppler imaging scanning, and control the probe 20 to receive ultrasonic echo signals which will be processed by the Doppler imaging processing device 50.

The Doppler imaging processing device 50 may analyze Doppler information of the ultrasonic echo signals to obtain information for Doppler imaging of the target tissue. In one embodiment, the information for Doppler imaging of the target tissue may include at least motion direction and/or velocity information of the fluid in the target tissue. For example, in the case that the target tissue is vascular tissue, the information for Doppler imaging of the vascular tissue may include at least the direction and magnitude of the velocity of the blood.

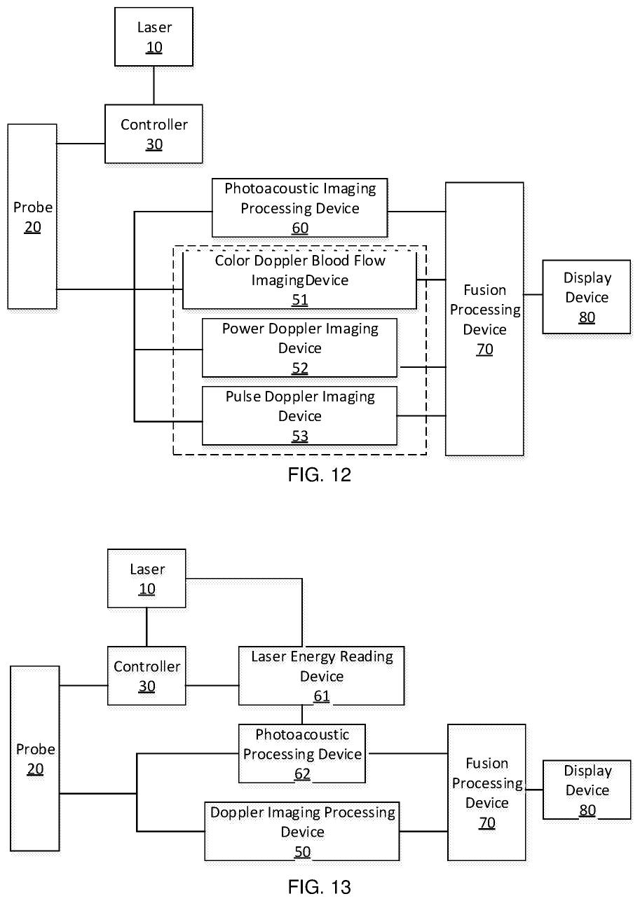

Referring to FIG. 12, in one embodiment, the Doppler imaging processing device 50 may include at least one of a color Doppler blood flow imaging device 51, an energy Doppler imaging device 52 and a pulse Doppler imaging device 53. It should be noted that in the embodiment shown in the figure all three mentioned above are included. The color Doppler blood flow imaging device 51 is a device which performs ultrasonic imaging using a Color Doppler Flow Image (CDFI) technique, and is used to process ultrasonic echo signals to obtain information for color Doppler flow imaging of the target tissue. The power Doppler imaging device 52 is a device which performs ultrasound imaging using Power Doppler Imaging (PDI) technique, and is used to process ultrasonic echo signals to obtain information for power Doppler imaging of the target tissue. The pulse Doppler imaging device 53 is a device which performs ultrasonic imaging using pulse-wave Doppler Imaging (PWDI) technique, and is used to process ultrasonic echo signals to obtain information for pulse Doppler imaging of the target tissue. Correspondingly, the information for Doppler imaging of the target tissue obtained by the Doppler imaging processing device 50 may include at least one of the information for color Doppler flow imaging of the target tissue, the information for power Doppler imaging of the target tissue and the information for pulse Doppler imaging of the target tissue.

The photoacoustic imaging processing device 60 may process the photoacoustic signals to obtain information for photoacoustic imaging of the target tissue. In one embodiment, the information for photoacoustic imaging of the target tissue may include at least location and/or morphological information of the target tissue. For example, in the case that the target tissue is vascular tissue, the information for photoacoustic imaging of the target tissue may include at least the location and/or morphological information of the blood vessel. In the photoacoustic imaging processing, the energy value of the emitted laser may need to be known. Since the energy value of each laser emission is different, the laser 10 may be set to record the energy value of the current laser emission after each laser emission is completed. Therefore, the energy value may be read after each time the laser emission is completed. For example, the controller 30 may notify when the energy value may be read.

Therefore, in one embodiment, referring to FIG. 13, the photoacoustic imaging processing device 60 may include a laser energy reading device 61 and a photoacoustic processing device 62. The laser 10 may record the energy value of each laser emission, and the laser energy reading device 61 may read the energy value of the each laser emitted by the laser 10. For example, the laser energy reading device 61 may read the energy values recorded by the laser 10 under the control of the controller 30. The photoacoustic processing device 62 may obtain the information for photoacoustic imaging of the target tissue according to the photoacoustic signals and the read energy values of the laser energy.

The fusion processing device 70 may generate an image of the target tissue according to at least the information for Doppler imaging of the target tissue obtained by the Doppler imaging processing device 50 and the information for photoacoustic imaging of the target tissue obtained by the photoacoustic imaging processing device 70.

The display device 80 may display the image of the target tissue generated by the fusion processing device 70.

The basic structure of the multi-modality imaging system of the present disclosure is described above, which may also include some other common structural components. For example, the multi-modality imaging system may further include an echo receiving processing device which processes the signals (such as photoacoustic signals and ultrasonic echo signals) received by the probe 20, such as filtering, amplifying, or beamforming, etc. The echo receiving processing device may send the processed signals to corresponding devices. For example, the photoacoustic signals may be processed and sent to the photoacoustic imaging processing device 60, and the ultrasonic echo signals obtained by the Doppler imaging scanning may be processed and sent to the Doppler imaging processing device 50.

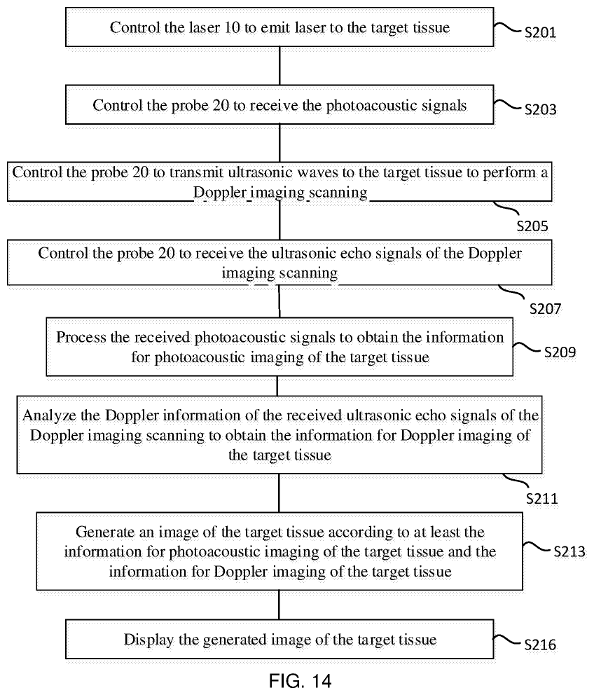

Referring to FIG. 14, in one embodiment, a multi-modality imaging method is provided, which may include steps S101 to S119. In one embodiment, the method may further include step S121. A more specific description will be presented below.

Step S201: controlling the laser 10 to emit laser to the target tissue.

Step S203: controlling the probe 20 to receive the photoacoustic signals.

Step S205: controlling the probe 20 to transmit ultrasonic waves to the target tissue to perform a Doppler imaging scanning.

Step S207: controlling the probe 20 to receive the ultrasonic echo signals of the Doppler imaging scanning.

Step S209: processing the photoacoustic signals received in step S203 to obtain the information for photoacoustic imaging of the target tissue. In one embodiment, the information for photoacoustic imaging of the target tissue may include at least location and/or morphological information of the target tissue. In one embodiment, step S209 may include controlling the laser 10 to record the energy value of each laser emission, reading the energy value of each laser emission of the laser 10 (for example, after the laser 10 emits a laser, the energy value of the current laser emission recorded by the laser 10 may be read), and obtaining the information for photoacoustic imaging of the target tissue according to the photoacoustic signals and the read energy value of the laser.

Step S211: analyzing the Doppler information of the ultrasonic echo signals of the Doppler imaging scan received in step S207 to obtain the information for Doppler imaging of the target tissue. In one embodiment, the information for Doppler imaging of the target tissue may include at least motion direction and/or velocity information of the fluid in the target tissue. In one embodiment, the information for Doppler imaging of the target tissue may include at least one of: the information for color Doppler flow imaging of the target tissue, the information for power Doppler imaging of the target tissue and the information for pulse Doppler imaging of the target tissue.

Step S213: generating an image of the target tissue according to at least the information for photoacoustic imaging of the target tissue and the information for Doppler imaging of the target tissue.

Step S215: displaying the image of the target tissue generated in step S213.

In the above steps, steps S2012 and S205 involve laser or ultrasonic emission, and steps S203 and S207 involve reception of photoacoustic signals or ultrasonic echo signals. In one embodiment, the sequence of these steps may be controlled. For example, the laser 10 may be controlled to emit lasers to the target tissue in a certain sequence, and the probe 20 may be controlled to transmit ultrasonic waves to the target tissue during an intermediate period between two adjacent laser emissions. For example, the laser 10 may be controlled to emit lasers to the target tissue, and the probe 20 may be controlled to receive the photoacoustic signals; and thereafter, the probe 20 may be controlled to transmit ultrasonic waves to the target tissue to perform Doppler imaging scanning, and receive the ultrasonic echo signals.

In one embodiment, the B-mode imaging processing device 40, the Doppler imaging processing device 50, the color Doppler blood flow imaging device 51, the energy Doppler imaging device 52, the pulse Doppler imaging device 53, the photoacoustic imaging processing device 60, the laser energy reading device 61, the photoacoustic processing device 62, the fusion processing device 70 and/or the echo receiving processing device 90 described above or any combination thereof may be implemented in one or more processors.

The multi-modality imaging system and method and the computer-readable storage medium of the embodiments above combine photoacoustic imaging with ultrasound Doppler imaging. When imaging a target tissue, the photoacoustic imaging is used to acquire the functional information of the target tissue and the ultrasound Doppler imaging is used to obtain the magnitude and direction of the flow velocity of the fluid in the target tissue. Not only the drawbacks of the photoacoustic imaging and ultrasound Doppler imaging can be overcome, but also their advantages can be combined, thereby providing users with more comprehensive and effective information about the target tissue.

This disclosure has been made with reference to various exemplary embodiments. However, those skilled in the art will recognize that changes and modifications may be made to the exemplary embodiments without departing from the scope of the present disclosure. For example, various operational steps, as well as components for carrying out operational steps, may be implemented in alternate ways depending upon the particular application or in consideration of any number of cost functions associated with the operation of the system, e.g., one or more of the steps may be deleted, modified, or combined with other steps.

The embodiments above may be implemented wholly or partially by software, hardware, firmware or any combination thereof. Additionally, as will be appreciated by one of ordinary skill in the art, principles of the present disclosure may be reflected in a computer program product on a computer-readable storage medium having computer-readable program code means embodied in the storage medium. Any tangible, non-transitory computer-readable storage medium may be utilized, including magnetic storage devices (hard disks, floppy disks, and the like), optical storage devices (CD-ROMs, DVDs, Blu-Ray discs, and the like), flash memory, and/or the like. These computer program instructions may be loaded onto a general purpose computer, special purpose computer, or other programmable data processing apparatus to produce a machine, such that the instructions that execute on the computer or other programmable data processing apparatus create means for implementing the functions specified. These computer program instructions may also be stored in a computer-readable memory that can direct a computer or other programmable data processing apparatus to function in a particular manner, such that the instructions stored in the computer-readable memory produce an article of manufacture, including implementing means that implement the function specified. The computer program instructions may also be loaded onto a computer or other programmable data processing apparatus to cause a series of operational steps to be performed on the computer or other programmable apparatus to produce a computer-implemented process, such that the instructions that execute on the computer or other programmable apparatus provide steps for implementing the functions specified.

While the principles of this disclosure have been shown in various embodiments, many modifications of structure, arrangements, proportions, elements, materials, and components, which are particularly adapted for a specific environment and operating requirements, may be used without departing from the principles and scope of this disclosure. These and other changes or modifications are intended to be included within the scope of the present disclosure.

The foregoing specification has been described with reference to various embodiments. However, one of ordinary skill in the art will appreciate that various modifications and changes can be made without departing from the scope of the present disclosure. Accordingly, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. As used herein, the terms “comprises,” “comprising,” and any other variation thereof, are intended to cover a non-exclusive inclusion, such that a process, a method, an article, or an apparatus that comprises a list of elements does not include only those elements but may include other elements not expressly listed or inherent to such process, method, system, article, or apparatus. Also, as used herein, the terms “coupled,” “coupling,” and any other variation thereof are intended to cover a physical connection, an electrical connection, a magnetic connection, an optical connection, a communicative connection, a functional connection, and/or any other connection.

Those having skill in the art will appreciate that many changes may be made to the details of the above-described embodiments without departing from the underlying principles of the invention. The scope of the present invention should, therefore, be determined only by the following claims.

We claim:

1. A multi-modality imaging system, comprising:
a laser which emits lasers to a target tissue;
a probe which perform transmission and reception of ultrasonic waves to and from the target tissue, wherein the reception of ultrasonic waves comprises receiving photoacoustic signals and receiving ultrasonic echo signals;
a controller which controls an emission sequence of the laser and a sequence for transmitting and receiving ultrasonic waves of the probe; and
a processor which:
processes the ultrasonic echo signals to obtain an information for B-mode imaging of the target tissue;
analyzes Doppler information of the ultrasonic echo signals to obtain an information for Doppler imaging of the target tissue;
processes the photoacoustic signals to obtain an information for photoacoustic imaging of the target tissue; and
generates an image of the target tissue according to at least the information for B-mode ultrasonic imaging of the target tissue obtained by the B-mode imaging processing device, the information for Doppler imaging of the target tissue obtained by the Doppler imaging processing device and the information for photoacoustic imaging of the target tissue obtained by the photoacoustic imaging processing device.

2. The multi-modality imaging system of claim 1, wherein the controller controls the laser to emit the lasers to the target tissue in a certain sequence and controls the probe to transmit the ultrasonic waves to the target tissue during an intermediate period between two adjacent laser emissions.

3. The multi-modality imaging system of claim 2, wherein:
the controller controls the laser to emit the lasers, and controls the probe to receive the photoacoustic signals; and then, the controller controls the probe to transmit ultrasonic waves for B-mode imaging scan, and controls the probe to receive ultrasonic echo signals of the B-mode imaging scan; and thereafter, the controller controls the probe to transmit ultrasonic waves for Doppler imaging scanning, and controls the probe to receive ultrasonic echo signals of the Doppler imaging scanning; or
the controller controls the laser to emit the lasers, and controls the probe to receive the photoacoustic signals; and then, the controller controls the probe to transmit ultrasonic waves for Doppler imaging scanning, and controls the probe to receive ultrasonic echo signals of the Doppler imaging scanning; and thereafter, the controller controls the probe to transmit ultrasonic waves for B-mode imaging scanning, and controls the probe to receive ultrasonic echo signals of the B-mode imaging scanning.

4. A multi-modality imaging system, comprising:
a laser which emits lasers to a target tissue;
a probe which perform transmission and reception of ultrasonic waves to and from the target tissue, wherein the reception of ultrasonic waves comprises receiving photoacoustic signals and receiving ultrasonic echo signals;
a controller which controls an emission sequence of the laser and a sequence for transmitting and receiving ultrasonic waves of the probe; and
a processor which:
analyzes Doppler information of the ultrasonic echo signals to obtain an information for Doppler imaging of the target tissue;
processes the photoacoustic signals to obtain an information for photoacoustic imaging of the target tissue; and
generates an image of the target tissue according to at least the information for Doppler imaging of the target tissue obtained by the Doppler imaging processing device and the information for photoacoustic imaging of the target tissue obtained by the photoacoustic imaging processing device.

5. The multi-modality imaging system of claim 4, wherein the controller controls the laser to emit the lasers to the target tissue in a certain sequence and controls the probe to transmit the ultrasonic waves to the target tissue during an intermediate period between two adjacent laser emissions.

6. The multi-modality imaging system of claim 5, wherein, the controller controls the laser to emit the lasers, and controls the probe to receive the photoacoustic signals; and thereafter, the controller controls the probe to transmit ultrasonic waves for Doppler imaging scanning, and controls the probe to receive ultrasonic echo signals of the Doppler imaging scanning.

7. The multi-modality imaging system of claim 4, wherein, the probe comprises an array of elements for converting electrical signals into ultrasonic waves and converting ultrasonic waves into electrical signals and a laser exit port; the probe transmits and receives ultrasonic waves by the array of elements, wherein receiving ultrasonic waves comprises receiving photoacoustic signals and receiving ultrasonic echo signals; and the laser exit port of the probe is connected to the laser through an optical fiber so as to transmit lasers emitted by the laser and transported by the optical fiber.

8. The multi-modality imaging system of claim 7, comprising two laser exit ports which are disposed on both sides of the probe, respectively.

9. The multi-modality imaging system of claim 4, wherein the information for Doppler imaging of the target tissue comprises at least motion direction and/or velocity information of a fluid in the target tissue.

10. The multi-modality imaging system of claim 4, wherein the information for photoacoustic imaging of the target tissue comprises at least location and/or morphological information of the target tissue.

11. The multi-modality imaging system of claim 4, wherein the processor:
reads an energy value of each laser emitted by the laser; and
obtains the information for photoacoustic imaging of the target tissue according to the photoacoustic signals and the read energy values of the laser.

12. The multi-modality imaging system of claim 4, further comprising a display device which displays the image of the target tissue generated by the fusion processing device.

13. A multi-modality imaging method, comprising:
emitting, by a laser, lasers to a target tissue;
receiving, by a probe, photoacoustic signals;
transmitting, by the probe, ultrasonic waves to the target tissue to perform a B-mode imaging scanning;
receiving, by the probe, ultrasonic echo signals of the B-mode imaging scanning;
transmitting, by the probe, ultrasonic waves to the target tissue to perform a Doppler imaging scanning;
receiving, by the probe, ultrasonic echo signals of the Doppler imaging scanning;
processing, by a processor, the photoacoustic signals to obtain an information for photoacoustic imaging of the target tissue;
processing, by the processor, the ultrasonic echo signals of the B-mode imaging scanning to obtain an information for B-mode ultrasonic imaging of the target tissue;
analyzing, by the processor, Doppler information of the ultrasonic echo signals of the Doppler imaging scanning to obtain an information for Doppler imaging of the target tissue; and
generating, by the processor, an image of the target tissue according to at least the information for photoacoustic imaging of the target tissue, the information for B-mode ultrasound imaging of the target tissue and the information for Doppler imaging of the target tissue.

14. The multi-modality imaging method of claim 14, wherein the lasers are emitted to the target tissue in a certain sequence and the ultrasonic waves are transmitted to the target tissue during an intermediate period between two adjacent laser emissions.

15. A multi-modality imaging method, comprising:
emitting, by a laser, lasers to a target tissue;
receiving, by a probe, photoacoustic signals;
transmitting, by the probe, ultrasonic waves to the target tissue to perform a Doppler imaging scanning;
receiving, by the probe, ultrasonic echo signals of the Doppler imaging scanning;
processing, by a processor, the photoacoustic signals to obtain an information for photoacoustic imaging of the target tissue;
analyzing, by the processor, Doppler information of the ultrasonic echo signals of the Doppler imaging scanning to obtain an information for Doppler imaging of the target tissue; and
generating, by the processor, an image of the target tissue according to at least the information for photoacoustic imaging of the target tissue and the information for Doppler imaging of the target tissue.

16. The multi-modality imaging method of claim 15, wherein the lasers are emitted to the target tissue in a certain sequence and the ultrasonic waves are transmitted to the target tissue during an intermediate period between two adjacent laser emissions.

17. The multi-modality imaging method of claim 15, wherein the information for Doppler imaging of the target tissue comprises at least one of: an information for color Doppler flow imaging of the target tissue, an information for power Doppler imaging of the target tissue and an information for pulse Doppler imaging of the target tissue.

18. The multi-modality imaging method of claim 15, wherein the information for Doppler imaging of the target tissue comprises at least motion direction and/or velocity information of a fluid in the target tissue.

19. The multi-modality imaging method of claim 15, wherein the information for photoacoustic imaging of the target tissue comprises at least location and/or morphological information of the target tissue.

20. The multi-modality imaging method of claim 15, wherein processing the photoacoustic signals to obtain information for photoacoustic imaging of the target tissue comprises:
reading an energy value of each laser emission; and
obtaining the information for photoacoustic imaging of the target tissue according to the photoacoustic signals and the read energy value of the laser.
